(19) United States
(12) Patent Application Publication
(10) Pub. No.: US 2024/0207262 A1
(43) Pub. Date: Jun. 27, 2024

(54) PROPHYLACTIC AND/OR THERAPEUTIC AGENT FOR AMYOTROPHIC LATERAL SCLEROSIS

(71) Applicant: NATIONAL UNIVERSITY CORPORATION TOKAI NATIONAL HIGHER EDUCATION AND RESEARCH SYSTEM, Nagoya-shi, Aichi (JP)

(72) Inventors: Masahisa KATSUNO, Nagoya-shi, Aichi (JP); Daisuke ITO, Nagoya-shi, Aichi (JP); Yohei IGUCHI, Nagoya-shi, Aichi (JP)

(21) Appl. No.: 18/557,236

(22) PCT Filed: Apr. 26, 2022
(86) PCT No.: PCT/JP2022/018823

§ 371 Date: Oct. 25, 2023

(30) Foreign Application Priority Data
Apr. 30, 2021 (JP) 2021-077588

(51) Int. Cl. A61K 31/501; A61P 25/28

(52) U.S. Cl. CPC A61K 31/501; A61P 25/28

(57) ABSTRACT

The present invention provides a novel prophylactic and/or therapeutic agent for amyotrophic lateral sclerosis. Provided is a prophylactic and/or therapeutic agent for amyotrophic lateral sclerosis.

### TECHNICAL FIELD

The present invention relates to a prophylactic and/or therapeutic agent for amyotrophic lateral sclerosis.

### BACKGROUND ART

PTL 1 discloses a pharmaceutical composition containing a therapeutically effective amount of a compound N-pyridazin-3-yl-4-(3-{[5-(trifluoromethyl) pyridin-2-yl]oxy}benzylidene) piperidine-1-carboxamide or a pharmaceutically acceptable salt thereof for the treatment of pain, urinary incontinence, overactive bladder, emesis, cognitive disorder, anxiety, depression, sleep disorder, eating disorder, movement disorder, glaucoma, psoriasis, multiple sclerosis, cerebrovascular disorder, brain injury, gastrointestinal disorder, hypertension, rheumatoid arthritis, osteoarthritis, or cardiovascular diseases.

### CITATION LIST

### Patent Literature

PTL 1: U.S. Pat. No. 4,434,313

### SUMMARY OF INVENTION

### Technical Problem

The present invention provides a novel prophylactic and/or therapeutic agent for amyotrophic lateral sclerosis (ALS).

### Solution to Problem

The present inventors conducted extensive research and found that a composition containing N-pyridazin-3-yl-4-(3-{[5-(trifluoromethyl) pyridin-2-yl]oxy}benzylidene) piperidine-1-carboxamide or a pharmaceutically acceptable salt thereof is useful in the prevention and/or treatment of amyotrophic lateral sclerosis.

Specifically, the present invention relates to the following prophylactic and/or therapeutic agent for amyotrophic lateral sclerosis.
Item 1. A prophylactic and/or therapeutic agent for amyotrophic lateral sclerosis, comprising N-pyridazin-3-yl-4-(3-{[5-(trifluoromethyl) pyridin-2-yl]oxy}benzylidene) piperidine-1-carboxamide or a pharmaceutically acceptable salt thereof.
Item 2. The prophylactic and/or therapeutic agent according to Item 1, wherein the amyotrophic lateral sclerosis is sporadic or familial.

### Advantageous Effects of Invention

The present invention provides a novel prophylactic and/or therapeutic agent for amyotrophic lateral sclerosis.

### BRIEF DESCRIPTION OF DRAWINGS

FIG. 1 shows an outline of the present invention: ALS metabolome research.

FIG. 2 shows subject patients in the present invention.

FIG. 3 shows the protocol of drug screening using NSC34 cells according to the present invention.

FIG. 4 shows the results of a viability assay in vitro in the present invention.

FIG. 5 shows the in vitro experimental results of PF-04457845 in the present invention.

FIG. 6 shows a drug efficacy analysis using SOD1^{G93A} transgenic mice in the present invention.

FIG. 7 shows the results of survival analysis using PF-04457845 in the present invention.

FIG. 8 shows the results of motion analysis (PF-04457845) in the present invention.

FIG. 9 shows a ChAT staining (motor neurons) progression phase in the present invention.

FIG. 10 shows a ChAT staining (motor neurons) progression phase in the present invention.

FIG. 11 shows GFAP staining and Iba-1 staining in the present invention.

FIG. 12 shows the results of improved cell viability with PF-04457845 of the present invention, compared with other FAAH inhibitors in in vitro experiments.

### DESCRIPTION OF EMBODIMENTS

The invention is described in detail below.

However, embodiments are described for better understanding of the invention.

The embodiments are not intended to limit the invention unless otherwise specified.

### (1) Usefulness of the Present Invention

Amyotrophic lateral sclerosis (ALS) is a disease of unknown cause involving selective and progressive degeneration and loss of primary motor neurons (upper motor neurons) and secondary motor neurons (lower motor neurons), and generally develops after middle age. The progression of ALS is relatively rapid, and people with ALS usually die within two to five years without the use of a ventilator.

Of all cases of ALS, about 5% have a family history, which is referred to as “familial amyotrophic lateral sclerosis” (familial ALS). Research reports that about 20% of familial ALS cases have mutations in the genes of enzymes that process free radicals (ALS1).

About 95% of ALS cases are sporadic. The onset patterns and progression speed of patients with sporadic ALS vary widely; the pathophysiology, biomarkers, etc. to define these have not been identified.

ALS is classified into three types according to the pattern of onset: (1) upper-limb onset (typical form), in which muscle atrophy and weakness are mainly seen in the upper limbs with spasticity in the lower limbs; (2) bulbar onset (progressive bulbar palsy form), in which bulbar symptoms such as dysarthria and dysphagia are mainly seen; and (3) lower-limb onset (pseudopolyneuritic form), in which symptoms start to appear in the lower limbs, and the tendon reflex in the lower limbs is decreased or lost in the early stage, with secondary motor neuron disorders more pronounced. There are also further diversified forms of ALS, such as cases involving respiratory paralysis that comes to the foreground from the beginning, cases involving predominant trunk muscle disorders, and cases accompanied by dementia.

Currently, there is no effective treatment for ALS. Although riluzole (oral drug) and edaravone (injectable drug) have been approved by regulatory authorities, their efficacy is very limited. In Japan, ALS is a designated intractable disease that was acknowledged as a specified disease in 1974.

The present invention relates to a novel prophylactic and/or therapeutic agent for amyotrophic lateral sclerosis (ALS). The prophylactic and/or therapeutic agent for ALS of the present invention is typically administered systemically or topically in the form of an oral or parenteral agent.

(2) Prophylactic and/or Therapeutic Agent for Amyotrophic Lateral Sclerosis (ALS)

The prophylactic and/or therapeutic agent for ALS of the present invention contains N-pyridazin-3-yl-4-(3-{[5-(trifluoromethyl) pyridin-2-yl]oxy}benzylidene) piperidine-1-carboxamide or a pharmaceutically acceptable salt thereof.

N-pyridazin-3-yl-4-(3-{[5-(trifluoromethyl) pyridin-2-yl]oxy}benzylidene) piperidine-1-carboxamide (another name: PF-04457845, English name: N-pyridazin-3-yl-4-(3-{[5-(trifluoromethyl) pyridin-2-yl]oxy}benzylidene) piperidine-1-carboxamide) is a compound with the following structure.

The originator is Pfizer, Inc.

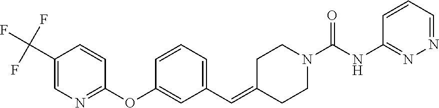 [SMILES: O=C(Nc1cccnn1)N1CCC(=Cc2cccc(Oc3ccc(C(F)(F)F)cn3)c2)CC1]

In the present invention, ALS is preferably sporadic or familial.

N-pyridazin-3-yl-4-(3-{[5-(trifluoromethyl) pyridin-2-yl]oxy}benzylidene) piperidine-1-carboxamide (PF-04457845) can form a pharmaceutically acceptable complex, salt, solvate, or hydrate.

The salt preferably includes acid addition salts (including diacids) and basic salts.

The pharmaceutically acceptable acid addition salts preferably include salts derived from inorganic acids, such as hydrochloric acid, nitric acid, phosphoric acid, sulfuric acid, hydrobromic acid, hydroiodic acid, hydrofluoric acid, and phosphorus acid; and salts derived from organic acids, such as aliphatic mono- and di-carboxylic acids, phenyl-substituted alkanoic acids, hydroxyalkanoic acids, alkanedioic acids, aromatic acids, aliphatic and aromatic sulfonic acids.

Such salts include acetate, adipate, aspartate, benzoate, besilate, bicarbonate, carbonate, bisulfate, sulfate, borate, cansylate, citrate, cyclamate, edisylate, esylate, formate, fumarate, gluceptate, gluconate, glucuronate, hexafluorophosphate, hibenzate, hydrochloride, chloride, hydrobromide, bromide, hydroiodide, iodide, isethionate, lactate, malate, maleate, malonate, mesylate, methyl sulfate, naphthylate, 2-napsylate, nicotinate, nitrate, orotate, oxalate, palmitate, pamoate, phosphoric acid, hydrogen phosphate, dihydrogen phosphate, pyroglutamate, saccharinate, stearate, succinate, tannate, tartrate, tosylate, trifluoroacetate, and xinafoate.

The pharmaceutically acceptable basic salts include metal cations, such as alkali metal or alkaline earth metal cations; and salts derived from bases, including amines.

Examples of appropriate metal cations include sodium (Na⁺), potassium (K⁺), magnesium (Mg²⁺), calcium (Ca²⁺), zinc (Zn²⁺), and aluminum (Al³⁺).

Examples of appropriate amines include arginine, N, N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethy-propane-1,3-diol, and procaine.

Because the prophylactic and/or therapeutic agent for ALS of the present invention contains N-pyridazin-3-yl-4-(3-{[5-(trifluoromethyl) pyridin-2-yl]oxy}benzylidene) piperidine-1-carboxamide or a pharmaceutically acceptable salt thereof, the prophylactic and/or therapeutic agent is useful in the prevention and/or treatment of ALS.

(3) Form for Administration of Prophylactic and/or Therapeutic Agent for Amyotrophic Lateral Sclerosis (ALS)

The prophylactic and/or therapeutic agent for ALS of the present invention is typically administered systemically or topically in the form of an oral or parenteral agent. Examples of oral agents include liquid agents for internal use (e.g., elixirs, syrups, and pharmaceutically acceptable aqueous agents, suspensions, and emulsions), solid agents for internal use (e.g., tablets, including sublingual tablets and orally disintegrating tablets, pills, capsules, including hard capsules, soft capsules, gelatin capsules, and microcapsules), powdered drugs, granule drugs, and troches.

Examples of parenteral agents include liquid agents (e.g., injectable drugs, such as subcutaneously injectable drugs, intravenously injectable drugs, intramuscularly injectable drugs, intraperitoneally injectable drugs, and intravenous drip), ophthalmic solutions (e.g., aqueous ophthalmic solutions, such as aqueous ophthalmic solutions, aqueous suspended ophthalmic solutions, viscous ophthalmic solutions, and solubilized ophthalmic solutions, non-aqueous ophthalmic solutions, such as non-aqueous ophthalmic solutions and non-aqueous suspended ophthalmic solutions), agents for external use (e.g., ointments, such as ophthalmic ointments), ear drops, and patches.

The agent may be a controlled-release formulation, such as a fast-release formulation or a sustained-release formulation.

The agent can be produced according to a known method, such as those described in the Japanese Pharmacopoeia.

A liquid agent for internal use is produced, for example, by dissolving, suspending, or emulsifying an active ingredient in a commonly used diluent (e.g., purified water, ethanol, or a mixture thereof).

The liquid agent may contain a wetting agent, a suspending agent, an emulsifier, a sweetener, a flavoring agent, an aromatic agent, a preservative, a buffer, etc.

A solid agent for internal use is formulated, for example, by mixing an active ingredient with an excipient (e.g., lactose, mannitol, glucose, microcrystalline cellulose, and starch), a binder (e.g., hydroxypropyl cellulose, polyvinyl pyrrolidone, and magnesium aluminometasilicate), a disintegrant (e.g., calcium cellulose glycolate), a lubricant (e.g., magnesium stearate), a stabilizer, a solubilizing agent (glutamic acid, and aspartic acid), etc., according to an ordinary method.

The solid agent for internal use may optionally be coated with a coating agent (e.g., sucrose, gelatin, hydroxypropyl cellulose, and hydroxypropyl methylcellulose phthalate), and may be coated with two or more layers.

An agent for external use (parenteral agents, such as ointments) is produced according to a known method or commonly used formula.

An ointment is produced by grinding or melting an active ingredient into a base.

The ointment base is selected from known or commonly used bases. The ointment base for use may be, for example, one, or a mixture of two or more selected from the following: higher fatty acids or higher fatty acid esters (e.g., adipic acid, myristic acid, palmitic acid, stearic acid, oleic acid, adipic acid ester, myristic acid ester, palmitic acid ester, stearic acid ester, and oleic acid ester), wax (e.g., beeswax, whale wax, and ceresin), surfactants (e.g., polyoxyethylene alkyl ether phosphoric ester), higher alcohols (e.g., cetanol, stearyl alcohol, and cetostearyl alcohol), silicon oils (e.g., dimethyl polysiloxane), hydrocarbons (e.g., hydrophilic petrolatum, white petrolatum, purified lanolin, and liquid paraffin), glycols (e.g., ethylene glycol, diethylene glycol, propylene glycol, polyethylene glycol, and macrogol), vegetable oils (e.g., castor oil, olive oil, sesame oil, and turpentine oil), animal oils (e.g., mink oil, egg yolk oil, squalane, and squalene), water, absorption promoters, and anti-rash agents. The ointment base may further contain a moisturizer, a preservative, a stabilizer, an antioxidant, a perfuming agent, etc.

The injectable drugs (parenteral agents) include solutions, suspensions, emulsions, and solid injectable drugs that are dissolved or suspended in a solvent in use.

An injectable drug is used, for example, by dissolving, suspending, or emulsifying an active ingredient in a solvent.

The solvent for use is, for example, injectable distilled water, physiological saline, vegetable oils, alcohols such as propylene glycol, polyethylene glycol, and ethanol, and combinations thereof.

The injectable drug may contain, for example, a stabilizer, a solubilizing agent (e.g., glutamic acid, aspartic acid, and Polysorbate 80 (registered trademark)), a suspending agent, an emulsifier, a soothing agent, a buffer, and a preservative. Sterilization or aseptic manipulation is performed in the final process to produce injectable drugs.

Alternatively, a sterile solid agent, such as a freeze-dried product, may be produced, and the agent may be dissolved in a sterile injectable distilled water or a solvent before use.

The prophylactic and/or therapeutic agent for ALS of the present invention contains N-pyridazin-3-yl-4-(3-{[5-(trifluoromethyl) pyridin-2-yl]oxy}benzylidene) piperidine-1-carboxamide or a pharmaceutically acceptable salt thereof. When used in combination with other drugs for the above purposes, the prophylactic and/or therapeutic agent for ALS of the present invention is usually administered systemically or topically in the form of an oral or parenteral agent.

The dosage of the N-pyridazin-3-yl-4-(3-{[5-(trifluoromethyl) pyridin-2-yl]oxy}benzylidene) piperidine-1-carboxamide or pharmaceutically acceptable salt thereof for the prophylactic and/or therapeutic agent for ALS of the present invention varies according to age, body weight, symptoms, therapeutic effects, administration method, treatment time, etc.

The prophylactic and/or therapeutic agent for ALS of the present invention is typically administered orally at a dose of N-pyridazin-3-yl-4-(3-{[5-(trifluoromethyl) pyridin-2-yl]oxy}benzylidene) piperidine-1-carboxamide or a pharmaceutically acceptable salt thereof in a range of 1 ng to 1,000 mg per adult, per dose once to several times per day (e.g., a dose of 4 mg/day).

The prophylactic and/or therapeutic agent for ALS of the present invention is typically administered parenterally at a dose of N-pyridazin-3-yl-4-(3-{[5-(trifluoromethyl) pyridin-2-yl]oxy}benzylidene) piperidine-1-carboxamide or a pharmaceutically acceptable salt thereof in a range of 0.1 ng to 10 mg per adult, per dose once to several times per day or administered intravenously continuously over a period of 1 hour to 24 hours per day.

The dosage of the prophylactic and/or therapeutic agent for ALS of the present invention varies according to various conditions. Thus, a dosage that is less than the above dosages may be sufficient in some cases, while a dosage beyond the ranges described above may be necessary in other cases.

### Examples

The present invention is described in more detail with reference to the Examples below.

### Experimental Method

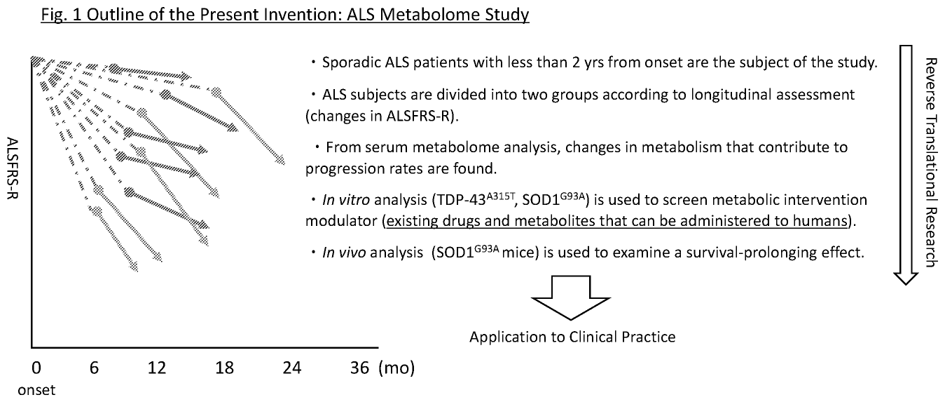

### (1) FIG. 1. Outline of the Present Invention: ALS Metabolome Study

In the present invention, a metabolome study of amyotrophic lateral sclerosis (ALS) was conducted through reverse translational research. The present invention is intended to link the discoveries made in clinical research to diagnosis or treatment and to provide to basic researchers feedback on problems and questions raised in clinical practice concerning new diagnostic methods or new drugs produced in such settings.

The present invention is a practical research method that examines clinical problems from a clinical perspective, i.e., what symptoms patients actually present and how they are troubled, and conducts basic and clinical research from that perspective to derive results that can be applied in actual clinical practice.

Patients with sporadic ALS with less than 2 years from onset were the subject of the study. The subjects with ALS were divided into two groups according to a longitudinal assessment of motor function changes (changes in ALSFRS-R). Metabolic changes that contribute to progression rates were found from serum metabolome analysis. In vitro analysis (disease models with TDP-43^{A315T} and SOD1^{G93A} mutations) was used to screen for metabolic intervention modulators (existing drugs and metabolites that can be administered to humans). In vivo analysis (SOD1^{G93A} transgenic mice) was used to examine a survival-prolonging effect.

Clinical application was performed.

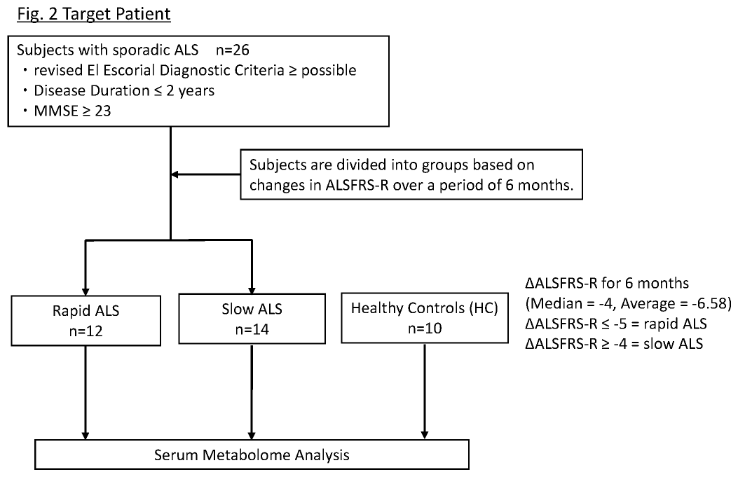

### (2) FIG. 2. Target Patient

FIG. 2 describes target patients.

Twenty-Six Patients with Sporadic ALS that Satisfy the Following Conditions
Rated “Possible” or higher in the diagnosis criteria of the revised El Escorial (Possible ALS)
With a disease duration of less than 2 years
Rated 23 or higher in the MMSE (Mini-Mental State Examination, a neuropsychological test performed when dementia is suspected)

The subjects were divided into groups based on changes in ALSFRS-R over a period of 6 months according to a method described in a document (Ohashi Y et al., Study of functional rating scale for amyotrophic lateral sclerosis: revised ALSFRS (ALSFRS-R), Japanese version. Brain and Nerve 2001; 53: 346-355).
(ΔALSFRS-R for 6 months, median=−4, average=−6.58)
Rapid ALS n=12 (patients with fast-progression ALS with ΔALSFRS-R≤−5=Rapid ALS)
Slow ALS n=14 (patients with slow-progression ALS with ΔALSFRS-R≥−4=Slow ALS)
Healthy controls (HC) n=10

Serum metabolome analysis was performed. The results of serum metabolome analysis indicated changes in several metabolites, including an increase in endocannabinoids, in the Rapid ALS group.

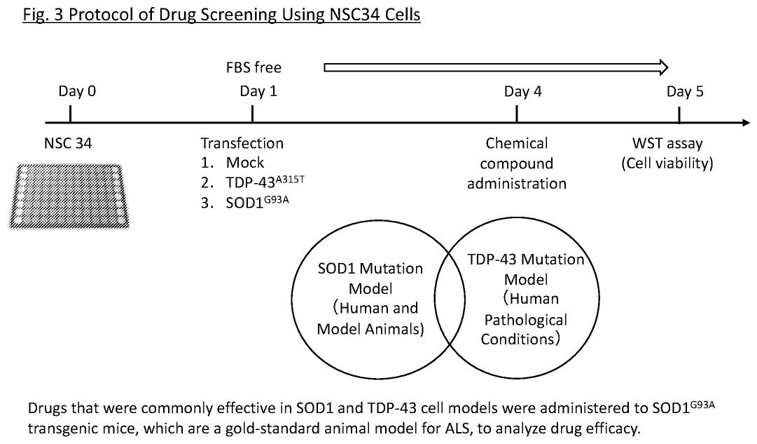

### (3) FIG. 3. Protocol of Drug Screening Using NSC34 Cells

A pathological cell model was created by transient forced expression of the following genes in NSC34 cells (mouse motor neuron) by transfecting the cells with a plasmid using Lipofectamine 2000 (R) (Thermo Fisher Scientific), and changes in cell viability due to drug administration were analyzed by WST assay.

The following describes a drug screening method.

### Drug Screening Method

Day 0: NSC 34 cell culture (DMEM FBS 10%)
Day 1: Transfection (Cells were transfected with a plasmid having the following genes inserted by using Lipofectamine 2000 (R) (Thermo Fisher Scientific).)
1. Mock (unmutated cells: control plasmid-transfected cells)
2. TDP-43^{A315T}: TDP-43 mutation model (reflecting human pathological conditions)
3. SOD1^{G93A}: mutation SOD1 familial model (mutation common to humans and animal models)
Cell culture is continued afterward in FBS-free DMEM.
Day 4: Drug administration
Day 5: WST assay (Cell viability is measured to determine the effect of drug administration.)

Two types of cellular models were used to bridge the gap between SOD1 transgenic mouse models and sporadic ALS human pathological conditions (TDP-43 cell model).

Drugs that were commonly effective in the SOD1 cell models and TDP-43 cell models were administered to SOD1^{G93A} transgenic mice, which are a gold-standard animal model for ALS, to analyze drug efficacy.

In the manner above, intervening drugs were administered to multiple metabolic pathways and drug screening was performed. As a result of the analysis of drug efficacy, a drug, PF04457845, was determined to be optimal for both the SOD1 cell model and the TDP-43 cell model.

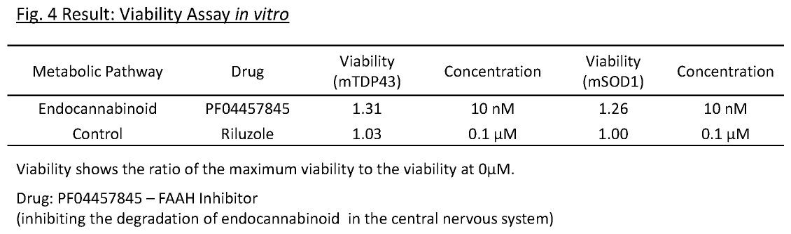

### (4) FIG. 4. Results: Viability Assay in Vitro

FIG. 4 shows the results of in vitro cell viability assay.

TABLE 1
Table 1

| Metabolic | | Viability | | Viability | |
|---|---|---|---|---|---|
| Pathway | Drug | (TDP-43^{A315T}) | Concentration | (SOD1^{G93A}) | Concentration |
| Endocannabinoids | PF04457845 | 1.31 | 10 nM | 1.26 | 10 nM |
| Control | Riluzole | 1.03 | 0.1 μM | 1.00 | 0.1 μM |

The viability indicates the ratio of maximum viability to viability at 0 μM. The endocannabinoid PF-04457845 yielded a viability about 1.3 times the viability with the existing drug riluzole (control) at a concentration one-tenth the concentration of riluzole.

For the drug, N-pyridazin-3-yl-4-(3-{[5-(trifluoromethyl) pyridin-2-yl]oxy}benzylidene) piperidine-1-carboxamide (another name: PF-04457845, English name: N-pyridazin-3-yl-4-(3-{[5-(trifluoromethyl) pyridin-2-yl]oxy}benzylidene) piperidine-1-carboxamide)) was selected.

N-pyridazin-3-yl-4-(3-{[5-(trifluoromethyl) pyridin-2-yl]oxy}benzylidene) piperidine-1-carboxamide is also referred below to as “PF-04457845.”

PF-04457845, also known as an FAAH (fatty acid amide hydrolase) inhibitor (inhibitor of FAAH), has functionality of inhibiting the degradation of endocannabinoids in the central nervous system or action of increasing the amount of endocannabinoids in the central nervous system.

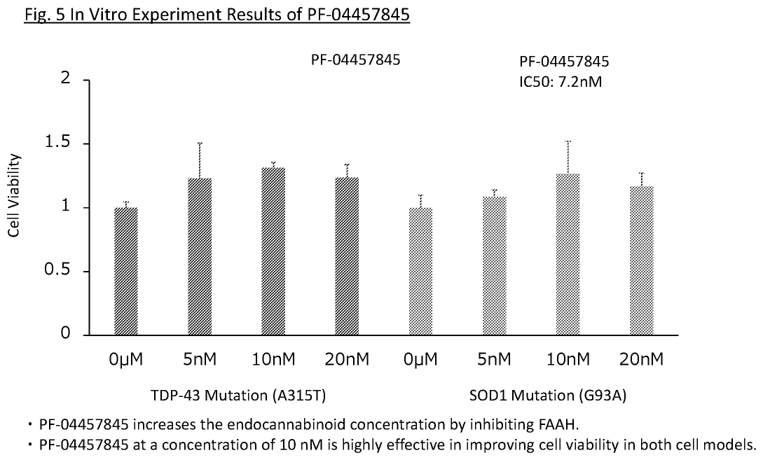

### (5) FIG. 5. In Vitro Experiment Results of PF-04457845

FIG. 5 shows the in vitro experiment results of PF-04457845.

The experimental conditions were those described in FIG. 3.

### PF-04457845, IC50: 7.2 nM

PF-04457845 has functionality of increasing the endocannabinoid concentration by inhibiting FAAH. PF-04457845 at a concentration of 10 nM was highly effective in improving the cell viability of both cell models of TDP-43 mutation (A315T) and SOD1 (G93A) mutation.

In the present invention, the administration of PF-04457845 provided a motor neuron protection effect, i.e., improved cellular activity (cell viability) of two types of ALS model cells.

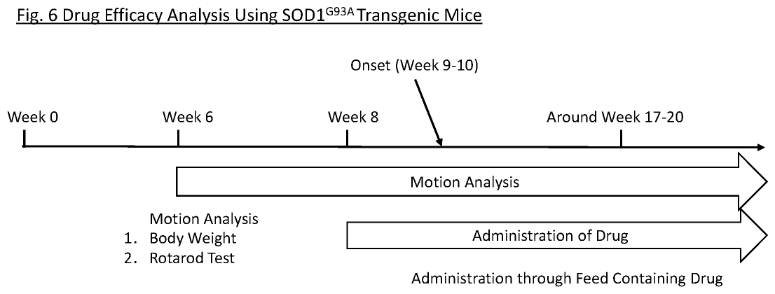

### (6) FIG. 6. Drug Efficacy Analysis Using SOD1^{G93A} Transgenic Mice

FIG. 6 shows drug efficacy analysis using SOD1^{G93A} transgenic mice.

PF-04457845 was mixed in the diet, and administration started at 8 weeks of age. A comparison was made with a group on a control diet that did not contain the drug. Drug efficacy analysis was conducted using body weight, motion analysis based on the Rotarod test (measured once a week), and survival analysis.

An ALS model animal with mutation of SOD1 gene, which is responsible for familial ALS, was used to conduct efficacy analysis of PF-04457845. The control was one that was free of the drug.

### Method of Drug Efficacy Analysis

Week 0:
Week 6: 1. Body weight, 2. Rotarod test (the method for measuring the motor function of mice)
Week 8: The drug (PF-04457845) was mixed with the diet, and administration started.
Week 9-10: Onset
Around week 17-20: Survival analysis

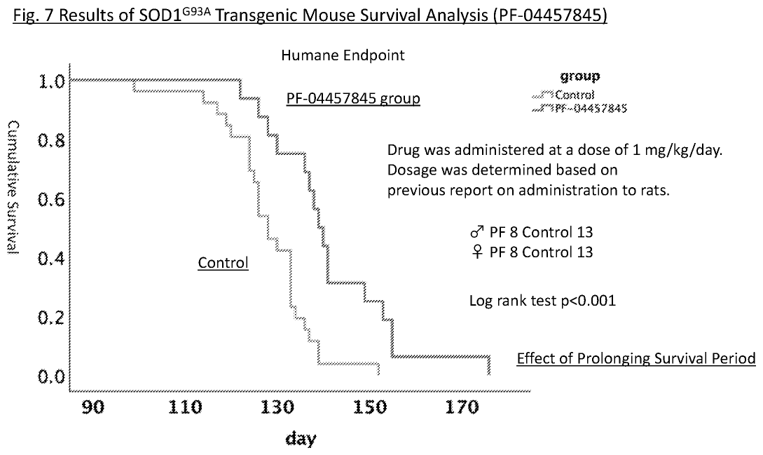

### (7) FIG. 7. Results of Mouse Survival Analysis (PF-04457845)

PF-04457845 is known as an FAAH inhibitor, and has functionality of increasing the endocannabinoid concentration in the central nervous system.

PF-04457845 was administered at a dose of 1 mg/kg/day. The dosage was determined based on a previous report on administration to rats (Ahn K et al. Journal of Pharmacology and Experimental Therapeutics 2011: 338; 114-124).

In accordance with the method shown in FIG. 6, survival analysis was performed.
♂ (male), PF-04457845: n=8, Control: n=13%
♀ (female), PF-04457845: n=8, Control: n=13
Log rank test p<0.001

Due to PF-04457845, an effect of prolonging the survival period was observed. PF-04457845 was confirmed to have a survival-prolonging effect on SOD1^{G93A} transgenic mice.

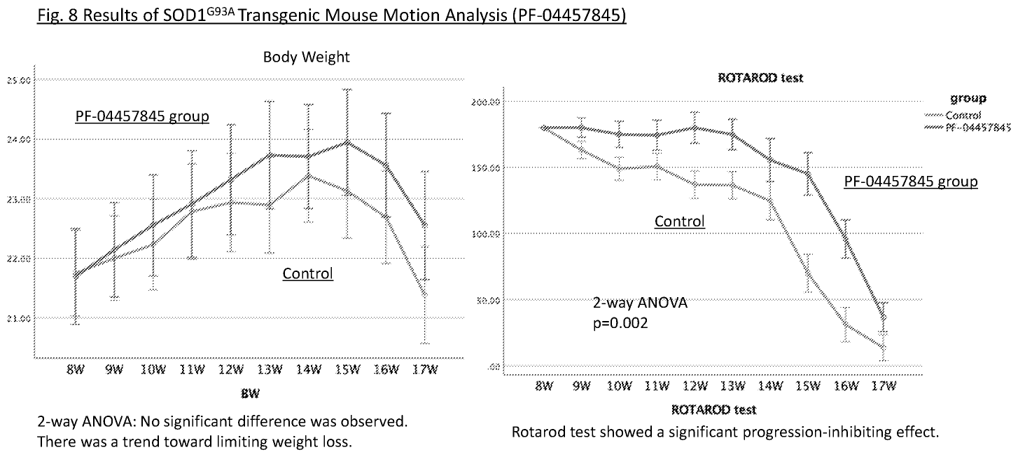

### (8) FIG. 8. Results of SOD1^{G93A} Transgenic Mouse Motion Analysis (PF-04457845)

FIG. 8 shows the results of motion analysis performed in accordance with the method shown in FIG. 6 (PF-04457845).

There was no significant difference in body weight (2-way ANOVA), but there was a trend toward limiting weight loss.

The Rotarod test (the method for measuring the motor function of mice) (2-way ANOVA, p=0.002) indicated a significant progression-inhibiting effect.

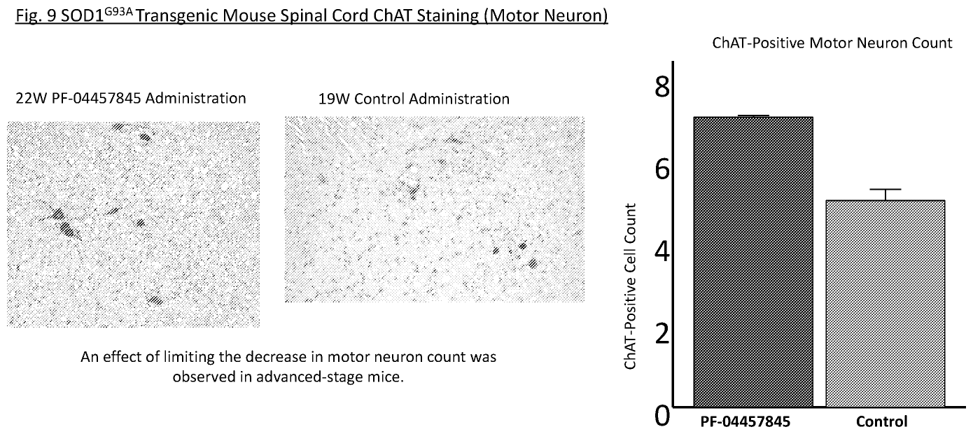

### (9) FIG. 9. SOD1^{G93A} Transgenic Mouse Spinal Cord ChAT Staining Change 1 in Mouse Spinal Cord Motor Neuron

In accordance with the method shown in FIG. 6, the drug was administered, and the pathological specimens of mouse spinal cords in an advanced stage of the disease were subjected to ChAT staining (motor neuron). FIG. 9 shows the results.

19W and 22W PF-04457845-administered mouse spinal cords were compared with a 19W control-administered mouse spinal cord.

The photographs are of a 22W PF-04457845-administered mouse spinal cord and a 19W control-administered mouse spinal cord. The graph shows the average ChAT-positive cell count of 22W PF-04457845-administered mouse spinal cords and the average ChAT-positive cell count of two specimens of 19W control-administered mouse spinal cords.

PF-04457845 administration was found to exhibit an effect of limiting the decrease in motor neuron count in advanced-stage mice as compared with control administration.

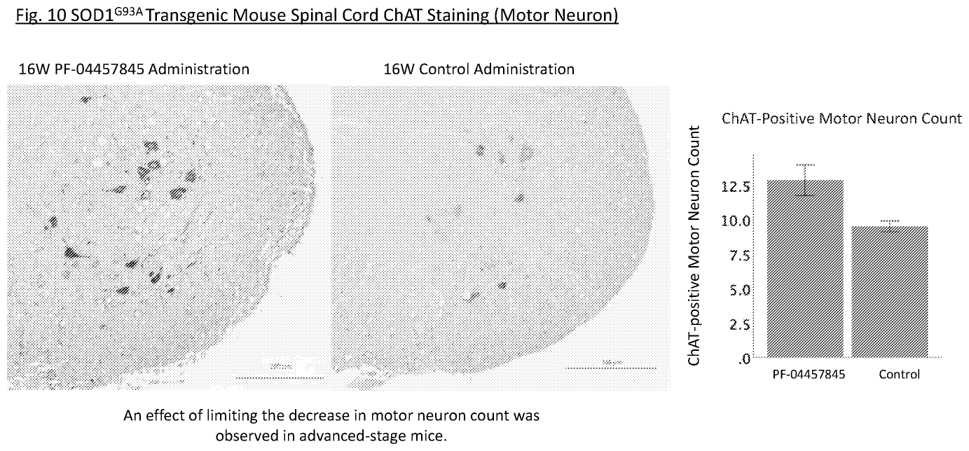

### (10) FIG. 10. SOD1^{G93A} Transgenic Mouse Spinal Cord ChAT Staining Change 2 in Mouse Spinal Cord Motor Neuron

In accordance with the method shown in FIG. 6, the drug was administered, and the pathological specimens of mouse spinal cords in an advanced stage of the disease were subjected to ChAT staining (motor neuron). FIG. 10 shows the results.

A 16W PF-04457845-administered mouse spinal cord was compared with a 16W control-administered mouse spinal cord.

The photographs are of the 16W PF-04457845-administered mouse spinal cord and 16W control-administered mouse spinal cord. The graph shows the average ChAT positive cell count of 16W PF-04457845-administered mouse spinal cords and the average ChAT-positive cell count of two specimens of 16W control-administered mouse spinal cords.

PF-04457845 administration was found to exhibit an effect of limiting the decrease in motor neuron count in advanced-stage mice as compared with control administration.

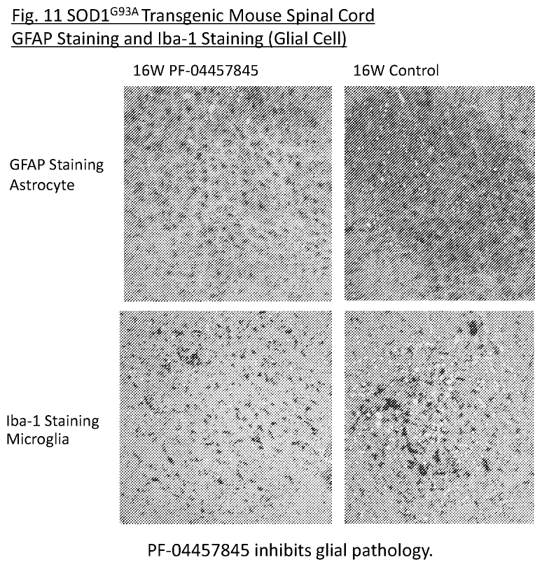

### (11) FIG. 11. SOD1^{G93A} Transgenic Mouse Spinal Cord GFAP Staining and Iba-1 Staining

FIG. 11 shows GFAP staining and Iba-1 staining of mouse spinal cords to which the drug was administered in accordance with the method shown in FIG. 6.

A 16W PF-04457845-administered mouse spinal cord was compared with a 16W control-administered mouse spinal cord.

GFAP staining indicated an effect of inhibiting the proliferation of astrocytes in PF-04457845-administered mice.

Iba-1 staining indicated an effect of inhibiting the proliferation of microglia in PF-04457845-administered mice.

PF-04457845 inhibited the proliferation of glial cells.

A previous report showed prolonged survival of CB₁-knockout SOD1^{G93A} transgenic mice, suggesting that CB₂ signaling that becomes dominant due to CB₁ receptor knockout inhibits the proliferation of glial cells and thus prolongs survival.

N-pyridazin-3-yl-4-(3-{[5-(trifluoromethyl) pyridin-2-yl]oxy}benzylidene) piperidine-1-carboxamide (PF-04457845) appears to have an additional effect on FAAH-inhibiting activity, and to have made CB₂ signaling dominant to inhibit motor neuron death and the proliferation of glial cells, thereby seemingly having prolonged survival.

### (12) Mouse Spinal Cord RNA Sequence

The experiment of PF-04457845 administration to SOD1^{G93A} transgenic mice was continued, and RNA sequencing of the spinal cord was performed on mice treated with a drug-infused diet and mice treated with a control diet, both at 16 weeks of age.

### Mouse Spinal Cord RNA Sequence Analysis: Analysis of GO (Gene Ontology)

GO analysis: This is a method for analyzing a group of genes that have many variable genes when genes are grouped according to their functionality among mRNAs whose RNA sequences show changes.

GO analysis indicated changes in a gene group associated with macrophage-microglia (glial cells being altered in sporadic ALS) and in a gene group associated with lipoproteins and lipid metabolism (lipid metabolism being altered in sporadic ALS).

Administration of PF-04457845 led to the differentiation of foam cells derived from macrophages and changes in a group of genes involved in low-density lipoprotein-based signaling pathways. In particular, the analysis revealed that administration of PF-04457845 increased, among these gene groups, the expression of CD36 and lipoprotein lipase (Lpl) and decreased the expression of Pla2g3 in mouse spinal cords.

### Mouse Spinal Cord RNA Sequence Analysis: IPA (Ingenuity® Pathway Analysis)

IPA analysis: An analysis to explore the association of known pathways, upstream factors, and toxicity with disease.

IPA analysis showed activation of IL-13 signaling pathway by administration of PF-04457845. IL-13 is an anti-inflammatory cytokine that induces differentiation of microglia into cytoprotective microglia (m2). The analysis revealed that microglia were induced to differentiate into m2 microglia via the IL-13 signaling pathway due to administration of PF-04457845. Administration of PF-04457845 is thought to have caused an anti-inflammatory effect and inhibited motor neuron degeneration.

The results of the analysis revealed that controlling macrophage-microglia differentiation and controlling lipid metabolism contributed in part to the efficacy of PF-04457845 in prolonging the survival from ALS and inhibiting motor function progression. Administration of PF-04457845 is also thought to have altered the expression of the gene group and brought about a survival-prolonging effect on SOD1 transgenic mice.

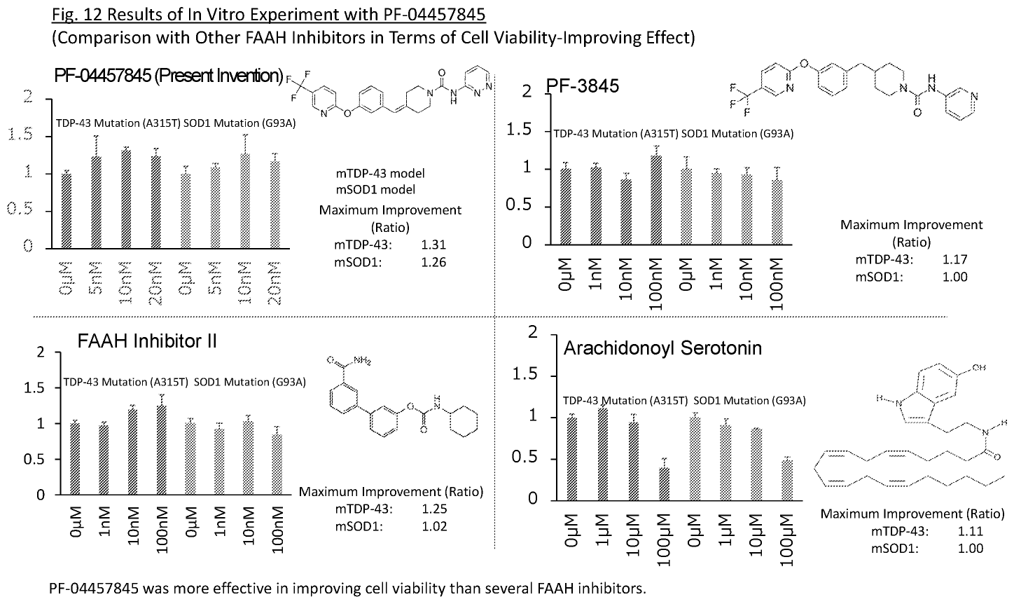

(13) FIG. 12. Results of In Vitro Experiment with PF-04457845

PF-04457845 (in vitro) was compared with other FAAH inhibitors. FIG. 12 shows the experiment results regarding an improving effect on cell viability.

The experimental conditions are those described in FIG. 3.

### PF-04457845, IC50: 7.2 nM

PF-04457845 was more effective in improving cell viability than several other FAAH inhibitors (PF-3845, arachidonoyl serotonin, FAAH inhibitor II).

### (14) Conclusion

From the Examples of the present invention, it is understood that the prophylactic and/or therapeutic agent for amyotrophic lateral sclerosis of the present invention is useful in the prevention and/or treatment of amyotrophic lateral sclerosis because the agent contains N-pyridazin-3-yl-4-(3-{[5-(trifluoromethyl) pyridin-2-yl]oxy}benzylidene) piperidine-1-carboxamide or a pharmaceutically acceptable salt thereof.

1. A prophylactic and/or therapeutic agent for amyotrophic lateral sclerosis, comprising N-pyridazin-3-yl-4-(3-{[5-(trifluoromethyl) pyridin-2-yl]oxy}benzylidene) piperidine-1-carboxamide or a pharmaceutically acceptable salt thereof.

2. The prophylactic and/or therapeutic agent according to claim 1, wherein the amyotrophic lateral sclerosis is sporadic or familial.
